(19) United States
(12) Patent Application Publication
(10) Pub. No.: US 2004/0261160 A1
(43) Pub. Date: Dec. 30, 2004

(54) Color diving mask and method of making same

(76) Inventor: Chih-Ban Hsu, Hsi Chih City (TW)

Correspondence Address: BIRCH STEWART KOLASCH & BIRCH, PO BOX 747, FALLS CHURCH, VA, 22040-0747, US

(21) Appl. No.: 10/602,715

(22) Filed: Jun. 25, 2003

(51) Int. Cl. A61F 9/02

(52) U.S. Cl. 2/439

(57) ABSTRACT

Color diving mask and method of manufacturing the same are disclosed. The mask comprises a frame formed of silicone rubber, a color skeletal element mounted in the frame, the skeletal element including a pair of rims, an integral glass lens fitted in the rims of the skeletal element, and an elastic strap attached to both sides of the frame. The method comprises fitting a glass lens in a color skeletal element, placing the color skeletal element in a mold, and integrally forming a frame in the mold by means of injection molding wherein the color skeletal element is mounted in the frame. The mask is aesthetic. Further, the mask is attractive to both young persons and fishes while diving.

### BACKGROUND OF THE INVENTION

1. Field of the Invention

The present invention relates to diving masks and more particularly to a color diving mask and a method of making same.

2. Description of Related Art

A conventional diving mask is shown in FIG. 5. The mask comprises a frame 50 formed of silicone rubber, a rim 51 in a forward side of the frame 50, a glass lens 60 fitted in the rim 51, and an elastic strap 70 attached to both sides of the frame 50. However, the prior diving mask suffered from a disadvantage. For example, the frame 50 is formed of transparent, colorless silicone rubber. As such, it is less attractive to young people who typically like color ones. Also, colorless diving masks are less attractive to fishes while diving. Hence, a need for improvement exists.

### SUMMARY OF THE INVENTION

It is an object of the present invention to provide a diving mask, comprising a frame formed of silicone rubber, the frame having a rim; a color skeletal element mounted in the rim, the color skeletal element being formed of plastic materials mixed with luminous materials, the color skeletal element including a pair of rims; an integral glass lens fitted in the rims of the color skeletal element; and an elastic strap attached to both sides of the frame.

It is another object of the present invention to provide a process of manufacturing a diving mask, comprising the steps of fitting a glass lens in a color skeletal element formed of plastic materials mixed with luminous materials; placing the color skeletal element in a mold; and integrally forming a frame in the mold by means of injection molding wherein the color skeletal element is mounted in the frame.

It is still another object of the present invention to provide a diving mask, comprising a frame formed of silicone rubber, the frame having two rims; a pair of color skeletal elements mounted in the rims respectively, each of the color skeletal elements being formed of plastic materials mixed with luminous materials; a pair of lenses fitted in the color skeletal elements; and an elastic strap attached to both sides of the frame.

The above and other objects, features and advantages of the present invention will become apparent from the following detailed description taken with the accompanying drawings.

### BRIEF DESCRIPTION OF THE DRAWINGS

FIG. 1 is an exploded view of a first preferred embodiment of diving mask according to the invention;

FIG. 2 is an assembled view of the FIG. 1 mask;

FIG. 3 is a perspective view of a second preferred embodiment of diving mask according to the invention;

FIG. 4 is a flow chart showing a sequence of method steps in manufacturing the FIG. 1 mask; and

FIG. 5 is a perspective view of a conventional diving mask.

### DETAILED DESCRIPTION OF THE PREFERRED EMBODIMENTS

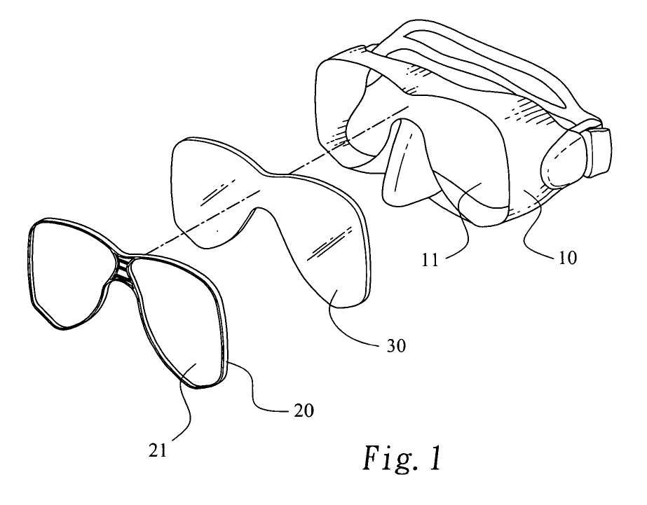

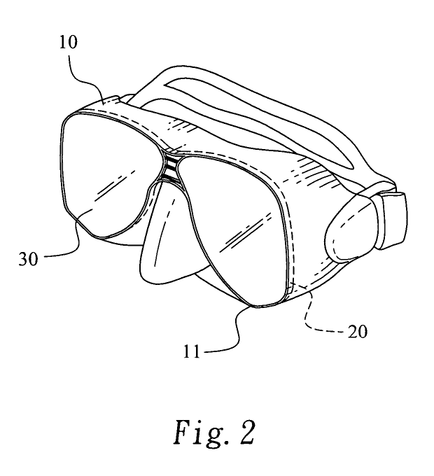

Referring to FIGS. 1 and 2, a diving mask constructed in accordance with the invention is shown. The mask comprises a frame 10 formed of silicone rubber, a rim 11 in a forward side of the frame 10, and a skeletal element 20 mounted in the rim 11. The skeletal element 20 is formed of color plastic materials, preferably, mixed with luminous materials so as to give off light in the dark, for example, sea water while diving. The color skeletal element 20 comprises a pair of rims 21. The mask further comprises an integral glass lens 30 fitted in the rims 21 and an elastic strap attached to both sides of the frame 10.

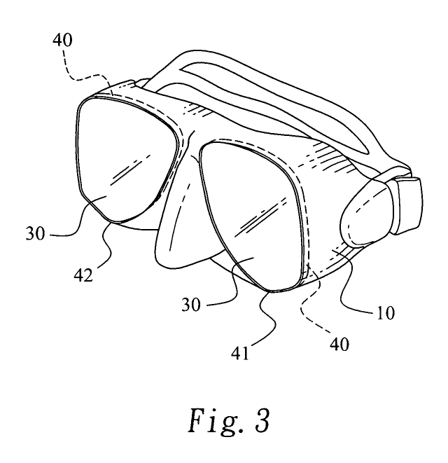

Referring to FIG. 3, there is shown a second preferred embodiment of diving mask according to the invention. The diving mask substantially has same structure as the first preferred embodiment. The differences between the first and the second preferred embodiments, i.e., the characteristics of the second preferred embodiment are detailed below. The color skeletal element 40 comprises two separate monocle rims 41 and 42 mounted in the rims 11 respectively. Also, a pair of glass lenses 30 are fitted in the rims 41 and 42 respectively. One rim 41 may have a color different from that of the other rim 42 so as to make the diving mask more colorful.

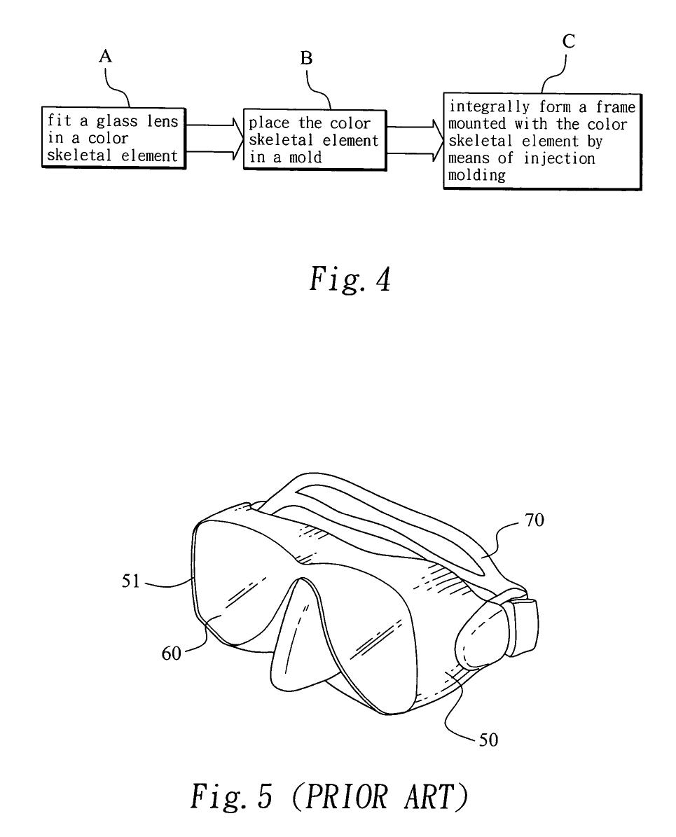

Referring to FIG. 4, a process of manufacturing the diving mask of the first preferred embodiment is illustrated. First, fit a glass lens 30 in the rims 21 of the color skeletal element 20 (step A). Next, place the color skeletal element 20 fitted with the glass lens 30 in a mold (step B). Finally, activate an injection molding machine to integrally form a frame 10 in the mold in which the color skeletal element 20 is mounted in the rim 11 of the frame 10 (step C).

The benefits of the invention include: The diving mask is colorful and aesthetic. Thus, it can attract young people to buy and even fishes while diving. Moreover, the lens is less likely to disengage from the rim while diving due to the integral structure of the mask. Also, the waterproof capability of the mask is much enhanced. Further, color skeletal element is durable since it is firmly enclosed by the frame.

While the invention herein disclosed has been described by means of specific embodiments, numerous modifications and variations could be made thereto by those skilled in the art without departing from the scope and spirit of the invention set forth in the claims.

What is claimed is:

1. A diving mask, comprising:
a frame formed of silicone rubber, the frame having a rim;
a color skeletal element mounted in the rim, the color skeletal element including a pair of rims;
an integral glass lens fitted in the rims of the color skeletal element; and
an elastic strap attached to both sides of the frame.

2. The diving mask of claim 1, wherein the color skeletal element is formed of plastic materials mixed with luminous materials.

3. A process of manufacturing a diving mask, comprising the steps of:
fitting a glass lens in a color skeletal element;
placing the color skeletal element in a mold; and
integrally forming a frame in the mold by means of injection molding wherein the color skeletal element is mounted in the frame.

4. The process of claim 3, wherein the color skeletal element is formed of plastic materials mixed with luminous materials.

5. A diving mask, comprising:
a frame formed of silicone rubber, the frame having two rims;
a pair of color skeletal elements mounted in the rims respectively;
a pair of lenses fitted in the color skeletal elements; and
an elastic strap attached to both sides of the frame.

6. The diving mask of claim 5, wherein one color skeletal element has a color different from that of the other color skeletal element.

7. The diving mask of claim 5, wherein each of the color skeletal elements is formed of plastic materials mixed with luminous materials.
